(19) United States
(12) Patent Application Publication
(10) Pub. No.: US 2007/0190079 A1
(43) Pub. Date: Aug. 16, 2007

(54) METHODS FOR THE SELECTIVE MODULATION OF PPAR

(75) Inventors: Andrew K. Shiau, San Diego, CA (US); Mark Eben Massari, San Diego, CA (US); Guy Oshiro, San Diego, CA (US); Ayman Kabakibi, San Diego, CA (US); James W. Malecha, San Diego, CA (US); Stewart A. Noble, San Diego, CA (US)

Correspondence Address: GLOBAL PATENT GROUP;ATTN: MS LAVERN HALL, P.O. BOX 38100, ST. LOUIS, MO, 63138, US

(73) Assignee: KALYPSYS, INC., San Diego, CA (US)

(21) Appl. No.: 11/687,949

(22) Filed: Mar. 19, 2007

Related U.S. Application Data
(63) Continuation-in-part of application No. 11/258,463, filed on Oct. 25, 2005.
(60) Provisional application No. 60/783,708, filed on Mar. 17, 2006.
(60) Provisional application No. 60/623,252, filed on Oct. 29, 2004.

(51) Int. Cl. A61K 31/495

(52) U.S. Cl. 424/278.1; 514/709; 514/255.02

(57) ABSTRACT

The present invention relates to methods of selective modulation of peroxisome proliferator activated receptors (PPARs) over G-protein coupled receptor 40 (GPR40), and the use of therapeutically effective amounts of compounds and pharmaceutical compositions which selectively modulate PPAR over GPR40 for the treatment of diseases in patients in need thereof. The methods disclosed herein are exceptionally useful in treating metabolic diseases whilst avoiding certain side effects common to modulators of PPAR previously disclosed in the art.

This application claims benefit of priority of U.S. provisional application No. 60/783,708, filed Mar. 17, 2006; this application is also a continuation-in-part of U.S. application Ser. No. 11/258,463, filed Oct. 25, 2005, pending, which itself claims the benefit of priority of U.S. provisional applications No. 60/623,252, filed Oct. 29, 2005, and 60/079,813, filed May 11, 2005, both now expired. The disclosures of all of these applications are hereby incorporated by reference as if written herein in their entireties.

### FIELD OF THE INVENTION

The present invention is directed to novel compositions and their application as pharmaceuticals for the treatment of disease. Methods of selective modulation of peroxisome proliferator activated receptor activity in a human or animal subject are also provided for the treatment of conditions such as obesity, insulin resistance, metabolic syndrome, and others in which a reduction in insulin resistance, an increase in glucose utilization, a reduction in visceral fat, a reduction in triglyceride (TG) levels, or an increase in levels of high-density lipoprotein (HDL), without induction or maintenance of a hypoglycemic state, is beneficial.

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUC1, PPAR-beta and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al., J Biol. Chem. 267:19051-19053 (1992); and Marcus et al., Proc. Natl. Acad. Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

The third subtype of PPAR, PPAR-delta (or alternatively, PPARδ, PPARβ, or NUC1) initially received much less attention than the other PPARs because of its ubiquitous expression and the unavailability of selective ligands. However, genetic studies and recently developed synthetic PPAR-δ agonists have helped reveal its role as a powerful regulator of fatty acid catabolism and energy homeostasis. Studies in adipose tissue and muscle have begun to uncover the metabolic functions of PPAR-δ. Transgenic expression of an activated form of PPAR-δ in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-γ target genes for lipid storage and lipogenesis remain unchanged. In parallel, PPAR-δ-deficient mice challenged with a high-fat diet show reduced energy uncoupling and are prone to obesity. Together, these data identify PPAR-δ as a key regulator of fat-burning, a role that opposes the fat-storing function of PPAR-γ. Thus, despite their close evolutionary and structural kinship, PPAR-y and PPAR-δ regulate distinct genetic networks. In skeletal muscle, PPAR-δ likewise upregulates fatty acid oxidation and energy expenditure, to a far greater extent than does the lesser-expressed PPAR-α (Evans R M et al 2004 Nature Med 1-7, 10 (4), 2004).

PPARδ is broadly expressed in the body and has been shown to be a valuable molecular target for treatment of dyslipidemia and other diseases. For example, in a recent study in insulin-resistant obese rhesus monkeys, a potent and selective PPARδ compound was shown to decrease VLDL and increase HDL in a dose response manner (Oliver et al., Proc. Natl. Acad. Sci. U.S.A. 98: 5305, 2001). Also, in a recent study in wild-type and HDL-lacking, ABCA1^{-/-} mice, a different potent and selective PPARδ compound was shown to reduce fractional cholesterol absorption in the intestine, and coincidently reduce expression of the cholesterol-absorption protein NPC1L1 (van der Veen et al., J. Lipid Res. 2005 46: 526-534).

Because there are three isoforms of PPAR and all of them have been shown to play important roles in energy homeostasis and other important biological processes in human body and have been shown to be important molecular targets for treatment of metabolic and other diseases (see Wilson, et al. J. Med. Chem. 43: 527-550 (2000)), it is desired in the art to identify compounds which are capable of interacting with multiple PPAR isoforms or compounds which are capable of selectively interacting with only one of the PPAR isoforms. Such compounds would find a wide variety of uses, such as, for example, in the treatment or prevention of obesity, for the treatment or prevention of diabetes, dyslipidemia, metabolic syndrome X and other uses.

Several PPAR-modulating drugs have been approved for use in humans. Fenofibrate and gemfibrozil are PPARα modulators; pioglitazone (Actos, Takeda Pharmaceuticals and Eli Lilly) and rosiglitazone (Avandia, GlaxcoSmithKline) are PPARγ modulators. Still other compounds are under development as PPAR drugs; among them are GW501516 (GlaxoSmithKhine, Ligand) and MCC-555 (netoglitazone, Mitsubishi Pharma). However, all of these compounds have liabilities as potential carcinogens, having been demonstrated to have proliferative effects leading to cancers of various types (colon; bladder with PPARα modulators and liver with PPARγ modulators) in rodent studies. Therefore, a need exists to identify other modulators of PPARs which lack these liabilities. Selective modulators of PPARδ may provide an opportunity for such improvements, and may even prove useful in the treatment of cancers, including colon, skin, and lung cancers.

Additionally, recent evidence points to a liability in these compounds as potential cross-activators of other proteins, such as the G-protein-coupled receptor GPR40. By way of background, GPR40 has recently been identified as a receptor for medium and long chain fatty acids (LCFAs) (Briscoe C P et al. (2003) J. Biol. Chem. 278, 11303-11311; Itoh Y et al. (2003) Nature 422, 173-176). GPR40 is expressed in the pancreas, monocytes, GI tract and brain (Briscoe, et al. 2003; Itoh et al. 2003). GPR40 couples to Gq and, therefore, receptor activation results in the elevation of intracellular calcium (Briscoe et al. 2003; Itoh et al. 2003). LCFAs can enhance glucose-stimulated insulin secretion (GSIS) in pancreatic β-cell lines (Haber E P et al. (2002) J. Cell Physiol. 194, 1-12; Itoh et al. 2003). Inhibition of GPR40 expression in a mouse insulinoma cell line blocks fatty acid-enhanced GSIS (Itoh et al. 2003). Mice deficient for GPR40 are resistant to high fat diet-induced hypertriglyceridemia, hyperglycemia, hyperinsulinemia, glucose intolerance, and hepatic steatosis (Steneberg P et al. (2005) Cell Metabol. 1, 245-258). In addition, GPR40 transgenic mice that specifically overexpress GPR40 in the pancreas develop diabetes (Steneberg et al. 2005). Taken together, these data suggest an important role for GPR40 in the regulation of insulin release and glucose homeostasis. Modulators of PPAR that do not activate or upregulate GPR40 would therefore be exceptionally useful in the treatment of metabolic diseases, including diabetes and obesity.

GPR40 is activated by the anti-diabetic thiazolidinediones rosiglitazone and MCC-555 (Kotarsky K et al. (2003) Biochem. Biophys. Res. Comm. 301, 406-410; Nilsson N E (2004) Ph.D. Thesis Lund University, Sweden, 1-102). A PPARδ-selective agonist, GW501516, can stimulate GSIS in isolated murine pancreatic islets in vitro (Tanaka T et al. (2003) Proc. Nat. Acad. Sci. 100, 15924-15929). These data suggest that GW501516 may also activate GPR40. Consistent with literature observations, the present invention confirms that rosiglitazone is a potent agonist of human GPR40, and in addition, that GW501516 also activates GPR40. In contrast, compounds disclosed herein are active against PPARs in the low nanomolar range, and do not activate GPR40 at concentrations up to the low micromolar range.

### SUMMARY OF THE INVENTION

A novel method for the selective modulation of PPAR over GPR40 has been discovered and is herein disclosed. Also disclosed is a novel method for treating PPAR-mediated disorders, especially metabolic disorders and related conditions, comprising the administration of a therapeutically effective amount of a compound which selectively modulates PPAR over GPR40, in a patient in need of such treatment.

Compounds and pharmaceutical compositions useful for the treatment of metabolic disorders which selectively modulate PPAR over GPR40 are disclosed, and their salts, esters, and prodrugs, together with methods of synthesizing and using the compounds. In broad aspect, therefore, the present invention provides for the entire class of said selective modulators of PPAR which do not activate or upregulate GPR40. The present invention also provides for pharmaceutical compositions comprising one or more compounds which selectively modulate PPAR over GPR40, together with at least one pharmaceutically acceptable diluent or carrier.

The present invention also provides methods of selectively modulating PPAR over GPR40 comprising contacting GPR40 with a compound as described herein.

The present invention also describes methods of treating a disease in a patient in need thereof comprising selectively modulating PPAR over GPR40. In certain embodiments, the disease to be treated by the methods of the present invention may be a metabolic disease. The present invention also provides for an embodiment wherein said method results in the elimination or reduction of one or more side effects typically associated with modulators of PPAR which are nonselective over GPR40. In certain embodiments, the side effect may be selected from the group consisting of hyperinsulinemia, hepatic steatosis, hypertriglyceridemia, and glucose intolerance.

PPAR modulators described herein may be modulating both PPARδ and PPARγ, or PPARα and PPARδ, or PPARα and PPARδ, or all three PPAR subtypes, or selectively modulating predominantly PPARδ, PPARα or PPARδ. Thus, the present invention provides for a method of selectively modulating PPAR over GPR40, comprising contacting said PPAR with a compound which does not activate GPR40. In certain embodiments, said modulation is also selective for PPARδ over PPARα and PPARγ. In further embodiments, said modulation of PPARδ is 100-fold selective or greater over said other isoforms. In yet further embodiments, said modulation is 200- to 500-fold selective over said other isoforms. In any of these embodiments, the PPAR modulator may be a compound of as described herein.

### DETAILED DESCRIPTION OF THE INVENTION

The present invention describes methods of treating a disease in a patient in need thereof comprising selectively modulating PPAR over GPR40.

In certain embodiments, said patient is a human.

In certain embodiments, said selectivity for PPAR over GPR40 is greater than or equal to 100-fold.

In certain embodiments, said selectivity for PPAR over GPR40 is greater than or equal to 1000-fold.

In a related embodiment, the present invention discloses a class of sulfonyl-substituted bicyclic compounds, useful as selective modulators of PPAR, defined by structural Formula I:

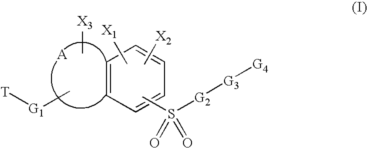

Or a salt, ester, or prodrug thereof, wherein;

A is a saturated or unsaturated hydrocarbon chain or a heteroatom-comprising hydrocarbon chain having from 3 to 5 atoms, forming a five- to seven-membered ring;

T is selected from the group consisting of —C(O)OH, —C(O)NH₂, and tetrazole;

G₁ is selected from the group consisting of —(CR¹R²)ₙ—, -Z(CR¹R²)ₙ—, —(CR¹R²)ₙZ-, —(CR¹R²)ᵣZ(CR¹R²)ₛ—;

Z is O, S or NR;

n is 0, 1, or 2;

r and s are independently 0 or 1;

R¹ and R² are independently selected from the group consisting of hydrogen, halo, optionally substituted lower alkyl, optionally substituted lower heteroalkyl, optionally substituted lower alkoxy, and lower perhaloalkyl or together may form an optionally substituted cycloalkyl;

X₁, X₂, and X₃ are independently selected from the group consisting of hydrogen, optionally substituted lower alkyl, optionally substituted cycloalkyl, halogen, perhaloalkyl, hydroxy, optionally substituted lower alkoxy, nitro, cyano, and NH₂;

G₂ is selected from the group consisting of a saturated or unsaturated cycloalkyl or heterocycloalkyl linker, optionally substituted with X₄ and X₅;

X₄ and X₅ are independently selected from the group consisting of hydrogen, optionally substituted lower alkyl, halogen, lower perhaloalkyl, hydroxy, optionally substituted lower alkoxy, nitro, cyano, NH₂, and CO₂R;

R is selected from the group consisting of optionally substituted lower alkyl and hydrogen;

G₃ is selected from the group consisting of a bond, a double bond, —(CR³R⁴)ₘ—, carbonyl, and —(CR³R⁴)ₘCR³═CR⁴—;

m is 0, 1, or 2;

R³ and R⁴ are independently selected from the group consisting of hydrogen, optionally substituted lower alkyl, optionally substituted lower alkoxy, optionally substituted aryl, lower perhaloalkyl, cyano, and nitro;

G₄ is selected from the group consisting of hydrogen, optionally substituted aryl, optionally substituted heteroaryl, optionally substituted cycloalkyl, optionally substituted cycloheteroalkyl, optionally substituted cycloheteroaryl, optionally substituted cycloalkenyl, and —N═(CR⁵R⁶); and

R⁵ and R⁶ are independently selected from the group consisting of hydrogen, optionally substituted aryl, optionally substituted heteroaryl, optionally substituted cycloalkyl, optionally substituted cycloalkenyl, and optionally substituted cycloheteroalkyl.

In further embodiments, the compound may be selected from the group consisting of the Examples described hereinbelow.

In yet further embodiments, said compound is selected from the group consisting of 4-[cis-2,6-dimethyl-4-(4-trifluoromethoxy-phenyl)-piperazine-1-sulfonyl]-indan-2-carboxylic acid and 4-[cis-2,6-dimethyl-4-(4-trifluoromethoxy-benzyl)-piperazine-1-sulfonyl]-indan-2-carboxylic acid.

In yet further embodiments, said compound is selected from the group consisting of (S)-4-[cis-2,6-dimethyl-4-(4-trifluoromethoxy-phenyl)-piperazine-1-sulfonyl]-indan-2-carboxylic acid tosylate and (S)-4-[cis-2,6-dimethyl-4-(4-trifluoromethoxy-benzyl)-piperazine-1-sulfonyl]-indan-2-carboxylic acid tosylate.

In another embodiment, the compound may be selected from the group consisting of those disclosed and described in PCT/US2004/010737, filed on Apr. 7, 2004; PCT/US2004/010889, filed on Apr. 7, 2004; PCT/US2004/010970, filed on Apr. 7, 2004; U.S. application Ser. No. 10/820,647, filed Apr. 7, 2004; PCT/US2004/043031, filed Dec. 20, 2004; PCT/US2005/011751 filed Apr. 7, 2005; U.S. application Ser. No. 11/102,356 filed Apr. 7, 2005; and U.S. application Ser. No. 11/258,463, filed Oct. 25, 2005; the contents of all of which are hereby incorporated by reference.

In yet another embodiment, the compound may be an analog of a known PPAR modulator, structurally modified to have selectivity over GPR40. In a further embodiment, the compound may be selected from the group consisting of glitazones, glitazars, and fibrates. In yet further embodiments, the glitazone may be selected from the group consisting of rosiglitazone, ciglitazone, pioglitazone, troglitazone, netoglitazone, and isaglitazone. In yet further embodiments, the glitazar may be selected from the group consisting of muraglitazar, naveglitazar, and tesaglitazar. In yet further embodiments, the fibrate may be selected from the group consisting of benzafibrate, clofibrate, ciprofibrate, etofibrate, fenofibrate, gemflbrozil, gefitinib, and GW5907354. In another embodiment, the compound may be selected from the group consisting of GW501516, MC-555, GSK677954, and GSK625019.

In further embodiments, said compound is administered to a human in a dose greater than or equal to 10 mg.

In yet further embodiments, said compound is administered to a human in a dose greater than or equal to 20 mg.

In yet further embodiments, said compound is administered to a human in a dose greater than or equal to 40 mg.

In yet further embodiments, said compound is administered to a human in a dose greater than or equal to 60 mg.

In yet further embodiments, said compound is administered to a human in a dose greater than or equal to 80 mg.

In yet further embodiments, said compound is administered to a human in a dose greater than or equal to 100 mg.

In further embodiments, said compound is administered to a human in a dose which is therapeutically effective.

In certain embodiments of this aspect, the present invention discloses methods: for raising HDL, lowering LDLc, shifting LDL particle size from small dense to normal LDL, lowering triglycerides, or inhibiting cholesterol absorption in a subject; for reducing insulin resistance, enhancing glucose utilization or lowering blood pressure in a subject; for reducing visceral fat in a subject; for reducing serum transaminases in a subject; or for treating disease; all comprising the administration of a therapeutic amount of a compound as described herein, to a patient in need thereof. In further embodiments, the disease to be treated may be a metabolic disease. In further embodiment, the metabolic disease may be selected from the group consisting of: obesity, diabetes, especially Type 2 diabetes, hyperinsulinemia, glucose intolerance, metabolic syndrome X, dyslipidemia, hypertriglyceridemia, hypercholesterolemia, and hepatic steatosis. In other embodiments, the disease to be treated may be selected from the group consisting of: cardiovascular diseases including vascular disease, atherosclerosis, coronary heart disease, cerebrovascular disease, heart failure and peripheral vessel disease; cancers including colon, skin, and lung cancers; inflammatory diseases including asthma, rheumatoid arthritis, and osteoarthritis; disorders associated with oxidative stress; inflammatory response to tissue injury; psoriasis, ulcerative colitis, dermatitis, and autoimmune diseases; polycystic ovary syndrome, climacteric, pathogenesis of emphysema, ischemia-associated organ injury, doxorubicin-induced cardiac injury, drug-induced hepatotoxicity, hypertoxic lung injury, scarring, wound healing, anorexia nervosa and bulimia nervosa. In preferred embodiments, the methods above do not result in the induction or maintenance of a hypoglycemic state.

In yet another aspect, the invention further contemplates compounds as disclosed herein or pharmaceutical compositions thereof for use in the manufacture of a medicament for the prevention or treatment of a disease or condition ameliorated by the selective modulation of PPAR over GPR40.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [C(O)H] and in combination is a —C(O)— group.

The term “carboxyl” or “carboxy,” as used herein, refers to C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to OH.

The term “hydroxyalkyl,” as used herein, alone or in combination, refers to a hydroxy group attached to the parent molecular moiety through an alkyl group.

The term “imino,” as used herein, alone or in combination, refers to ═N The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′ — group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substitutents of an “optionally substituted” group may include, without limitation, one or more substitutents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substitutents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃)—Where substitutents are recited without qualification as to substitution, both substituted and unsubstituted forms are encompassed. Where a substitutent is qualified as “substituted,” the substituted form is specifically intended. Additionally, different sets of optional substitutents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substitutent, and every term should be understood to be independent of every other in terms of selection from a group. Should any variable, substitutent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substitutents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

In the event that any element is designated to be “a bond,” what is meant is that said element collapses to a bond linking the elements on both its sides. For example, in Formula I above, when G₃ is designated to be “a bond”, the structure shown below (right side) is intended: the entity designated G₃ collapses to a single bond connecting G₂ and G₄: 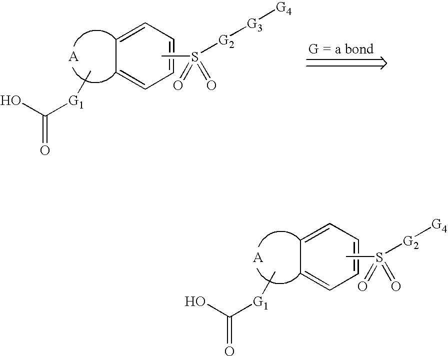
Similarly, when, within G₁, n is 0 or both r and s are 0, G₁ collapses to a bond connecting A and T.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “activate” refers to increasing the cellular function of a target enzyme or protein.

The term “inhibit” refers to decreasing the cellular function of a target enzyme or protein. The target enzyme or protein function may be the interaction with a natural binding partner or catalytic activity.

The term “modulate” refers to the ability of a compound of the invention to alter the function of a target enzyme or protein. A modulator may activate the activity of a target enzyme or protein. The term “modulate” also refers to altering the function of a target enzyme or protein by increasing or decreasing the probability that a complex forms between a target enzyme or protein and a natural binding partner. A modulator may increase the probability that such a complex forms between the target enzyme or protein and the natural binding partner, may increase or decrease the probability that a complex forms between the target enzyme or protein and the natural binding partner depending on the concentration of the compound exposed to the target enzyme or protein, and or may decrease the probability that a complex forms between the target enzyme or protein and the natural binding partner.

“PPAR modulator” is used herein to refer to a compound that exhibits an EC₅₀ with respect to PPAR activity of no more than about 100 μM and more typically not more than about 50 μM, as measured in the PPAR assay described generally hereinbelow. “EC₅₀” is that concentration of modulator which either activates or reduces the activity of an enzyme (e.g., PPAR) to half-maximal level. Representative compounds of the present invention have been discovered to exhibit modulatory activity against PPAR. Compounds of the present invention preferably exhibit an EC₅₀ with respect to PPAR of no more than about 10 μM, more preferably, no more than about 5 μM, even more preferably not more than about 1 μM, and most preferably, not more than about 200 nM, as measured in the PPAR assay described herein.

The term “selective” as used herein means having the characteristic or property of being highly specific in binding, activity, or effect. Compounds described herein as “selective for PPAR over GPR40,” for example, may preferentially bind and/or modulate PPAR in favor of GPR40. The degree of selectivity may vary, but preferably a selective compound would be at least tenfold selective for the desired target (e.g., PPAR). More preferably, the compound would be 100- to 1000-fold selective. Alternatively, a compound may be selective the sense of producing a differential effect. For example, such a compound may bind both PPAR and GPR40 with equal or similar affinity, but activate one while inhibiting the other.

The term “therapeutically effective amount” as used herein refers to that amount of the compound being administered which will relieve to some extent one or more of the symptoms of the disease, condition or disorder being treated.

The term “compound” is meant to be interchangeable with the term “active compound” or “drug,” and refers to a compound having beneficial prophylactic and/or therapeutic properties when administered to a patient and/or activity against a biological target which is associated with a disease affecting a patient.

The term “side effect,” as used herein is synonymous with “adverse effect,” and means any unintended, and undesirable, consequence of any kind of medical treatment in a patient. In the context of the present invention, this generally means an adverse drug reaction in a patient, and it may manifest as morbidity, mortality, alteration in body weight, levels of enzymes, loss of function, or as a pathological change detected at the microscopic, macroscopic or physiological level. It may also be indicated by symptoms reported by a patient. Adverse effects may cause a reversible or irreversible change, including an increase or decrease in the susceptibility of the individual to other chemicals, foods, or procedures (e.g. drug interaction). Such an effect might have the consequence of causing a patient to discontinue use of a drug due to discomfort or the development or worsening of a disease. Such an effect might also have the consequence of limiting the dosage of an otherwise effective drug, perhaps even to subtherapeutic levels. A side effect may be caused by interaction of a drug with an enzyme or other protein other than its target; alternatively, a drug may have an adverse effect outside of the target organ or tissue of interest. Examples of side effects in the context of the present invention include, without limitation, hyperinsulinemia, hepatic steatosis, hypertriglyceridemia, and glucose intolerance,

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The compounds disclosed herein can exist as therapeutically acceptable salts.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In certain embodiments, the salt may be selected from the sulfate, sodium, potassium, magnesium, calcium, hydrochloride, phosphonate, and tosylate salts of the compounds of Formula I. In further embodiments, the salt is the tosylate salt of a compound of Formula I.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5°% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastimes comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit. Specific, non-limiting examples of possible combination therapies include use of the compounds of the invention with: (a) statin and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone); and (c) antihypertensive agents such as angiotensin antagonists, e.g., telmisartan, calcium channel antagonists, e.g. lacidipine and ACE inhibitors, e.g., enalapril.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PPAR-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PPAR-mediated disorders.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

Compounds according to the present invention can be synthesized as described in: PCT/US2004/010737, filed on Apr. 7, 2004; PCT/US2004/010889, filed on Apr. 7, 2004; PCT/US2004/010970, filed on Apr. 7, 2004; U.S. application Ser. No. 10/820,647, filed Apr. 7, 2004; PCT/US2004/043031, filed Dec. 20, 2004; PCT/US2005/011751 filed Apr. 7, 2005; U.S. application Ser. No. 11/102,356 filed Apr. 7, 2005; and U.S. application Ser. No. 11/258,463, filed Oct. 25, 2005; U.S. Pat. No. 6,710,063, issued Mar. 23, 2004; U.S. Pat. No. 5,002,953, issued Mar. 26, 1991; U.S. Pat. No. 4,572,912, issued Feb. 25, 1986; U.S. Pat. No. 4,287,200, issued Sep. 1, 1981; U.S. Pat. No. 4,687,777, issued Aug. 18, 1987; United Kingdom Patent No. 2058785B, published Apr. 15, 1981; and PCT Application No. PCT/US97/01808, filed Jan. 31, 1997; the contents of all of which are hereby incorporated by reference.

The invention is further illustrated by the following examples, set forth below in Table 1.

TABLE 1

| | | IUPAC Name |
|---|---|---|
| Example | Structure | (and common/originator) |
| 1 | 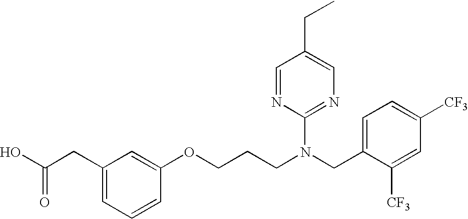 | 2-(3-(3-((2,4- bis(trifluoromethyl)benzyl)(5- ethylpyrimidin-2- yl)amino)propoxy)phenyl)acetic acid |
| 2 | 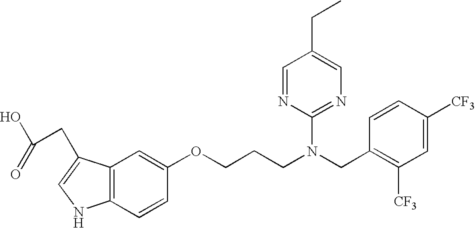 | 5-{3-[(2,4-Bis-trifluoromethyl- benzyl)-(5-ethyl-pyrimidin-2-yl)- amino]-propoxy}-1H-indol-3-yl)- acetic acid) |
| 3 | 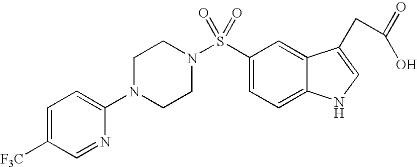 | 2-(6-(4-(5-(trifluoromethyl)pyridin-2- yl)piperazin-1-ylsulfonyl)-1H-indol- 1-yl)acetic acid |
| 4 | 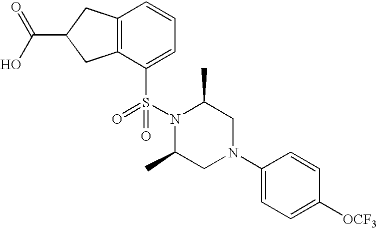 | 4-[cis-2,6-Dimethyl-4-(4- trifluoromethoxy-phenyl)-piperazine- 1-sulfonyl]-indan-2-carboxylic acid |
| 5 | One single stereoisomer of Example 4; R/S | |
| | unresolved. | |
| 6 | Corresponding other single stereoisomer of | |
| | Example 4; R/S. | |
| 7 | 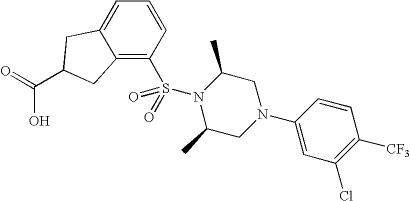 | 4-[4-(3-Chloro-4-trifluoromethyl- phenyl)-cis-2,6-dimethyl-piperazine- 1-sulfonyl]-indan-2-carboxylic acid |
| 8 | 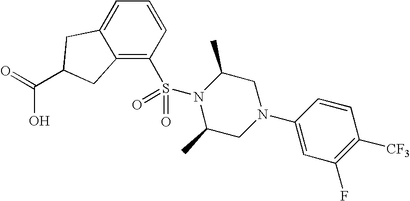 | 4-[4-(3-Fluoro-4-trifluoromethyl- phenyl)-cis-2,6-dimethyl-piperazine- 1-sulfonyl]-indan-2-carboxylic acid |
| 9 | 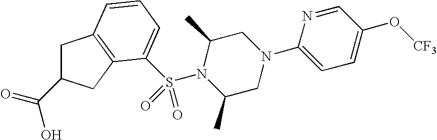 | 4-[cis-2,6-Dimethyl-4-(5- trifluoromethyl-pyridin-2-yl)- piperazine-1-sulfonyl]-indan-2- carboxylic acid |
| 10 | 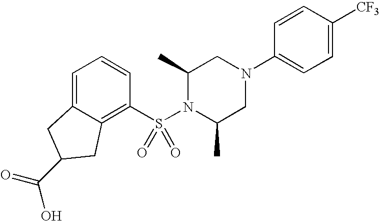 | 4-[cis-2,6-Dimethyl-4-(4- trifluoromethyl-phenyl)-piperazine-1- sulfonyl]-indan-2-carboxylic acid |
| 11 | 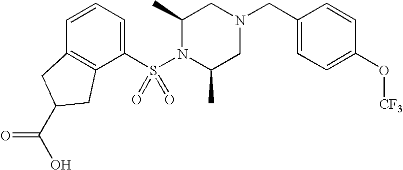 | 4-[cis-2,6-Dimethyl-4-(4- trifluoromethoxy-benzyl)-piperazine- 1-sulfonyl]-indan-2-carboxylic acid |
| 12 | 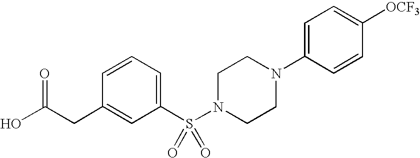 | {3-[4-(4-Trifluoromethoxy-phenyl)- piperazine-1-sulfonyl]-phenyl}-acetic acid |
| 13 | 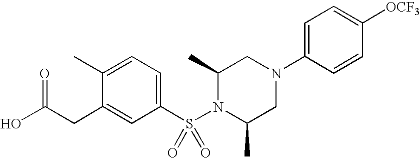 | 2-(5-(cis-2,6-Dimethyl-4-(4- (trifluoromethoxy)phenyl)piperazin- 1-ylsulfonyl)-2-methylphenyl)acetic acid |
| 14 | 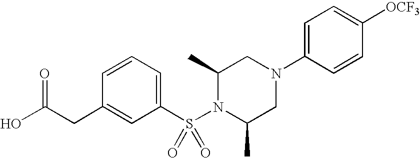 | {3-[2,6-Dimethyl-4-(4- trifluoromethoxy-phenyl)-piperazine- 1-sulfonyl]-phenyl}-acetic acid |
| 15 | 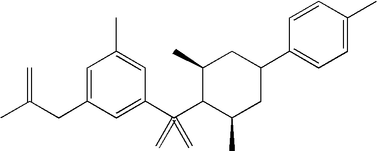 | {3-[2,6-Dimethyl-4-(4- trifluoromethoxy-phenyl)-piperazine- 1-sulfonyl]-5-methyl-phenyl}-acetic acid |
| 16 | 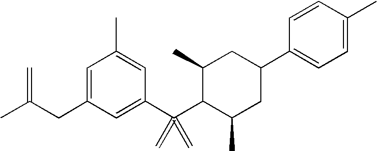 | {3-[2,6-Dimethyl-4-(4- trifluoromethoxy-phenyl)-piperazine- 1-sulfonyl]-5-trifluoromethyl- phenyl}-acetic acid |
| 17 | 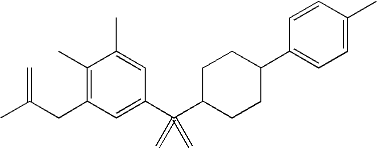 | {2-Bromo-3-methyl-5-[4-(5- trifluoromethyl-pyridin-2-yl)- piperazine-1-sulfonyl]-phenyl}-acetic acid |
| 18 | 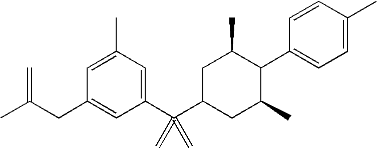 | 2-(3-(-3,5-Dimethyl-4-(5- (trifluoromethyl)pyridine-2- yl)piperazin-1-ylsulfonyl)-5- methylphenyl)acetic acid |
| 19 | 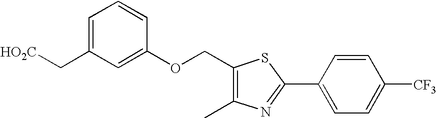 | {3-[4-Methyl-2-(4-trifluoromethyl- phenyl)-thiazol-5-ylmethoxy]- phenyl}-acetic acid |
| 20 | 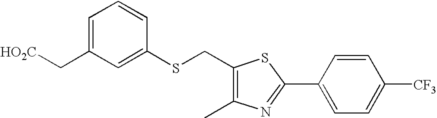 | {3-[4-Methyl-2-(4-trifluoromethyl- phenyl)-thiazol-5-ylmethylsulfanyl]- phenyl}-acetic acid |
| 21 | 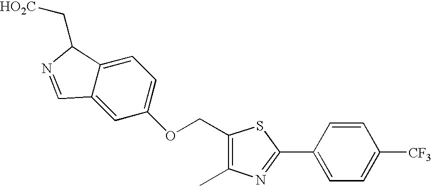 | {5-[4-Methyl-2-(4-trifluoromethyl- phenyl)-thiazol-5-ylmethoxy]-indol- 1-yl}-acetic acid |
| 22 | 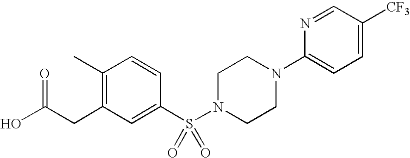 | {2-Methyl-5-[4-(5-trifluoromethyl- pyridin-2-yl)-piperazine-1-sulfonyl]- phenyl}-acetic acid |
| 23 | 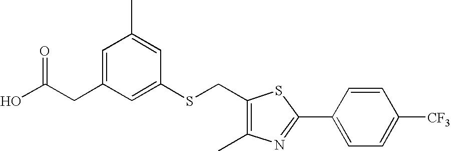 | {3-Methyl-5-[4-methyl-2-(4- trifluoromethyl-phenyl)-thiazol-5- ylmethylsulfanyl]-phenyl}-acetic acid |
| 24 | 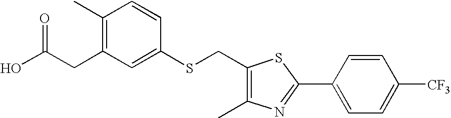 | {2-Methyl-5-[4-methyl-2-(4- trifluoromethyl-phenyl)-thiazol-5- ylmethylsulfanyl]-phenyl}-acetic acid |
| 25 | 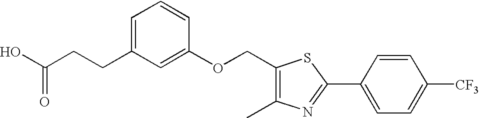 | 3-{3-[4-Methyl-2-(4-trifluoromethyl- phenyl)-thiazol-5-ylmethoxy]- phenyl}-propionic acid |
| 26 | 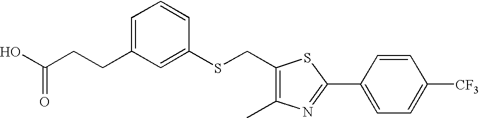 | 3-{3-[4-Methyl-2-(4-trifluoromethyl- phenyl)-thiazol-5-ylmethylsulfanyl]- phenyl}-propionic acid |
| 27 | 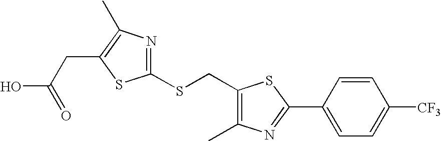 | {4-Methyl-2-[4-methyl-2-(4- trifluoromethyl-phenyl)-thiazol-5- ylmethylsulfanyl]-thiazol-5-yl}-acetic acid |
| 28 | 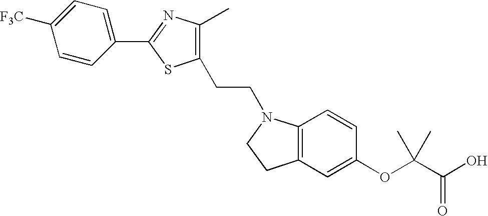 | 2-Methyl-2-(1-{2-[4-methyl-2-(4- trifluoromethyl-phenyl)-thiazol-5-yl]- ethyl}-2,3-dihydro-1H-indol-5- yloxy)-propionic acid |
| 29 | 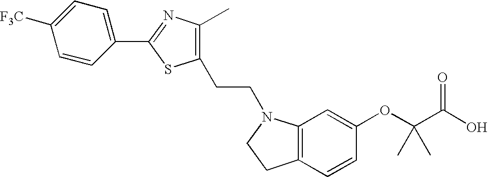 | 2-Methyl-2-(1-{2-[4-methyl-2-(4- trifluoromethyl-phenyl)-thiazol-5-yl]- ethyl}-2,3-dihydro-1H-indol-6- yloxy)-propionic acid |
| 30 | 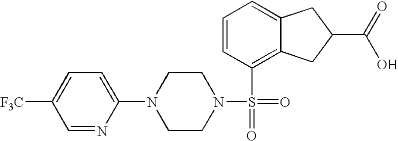 | 4-[4-(4-Trifluoromethyl-pyridin-2- yl)-piperazine-1-sulfonyl]-indan-2- carboxylic acid |
| 31 | 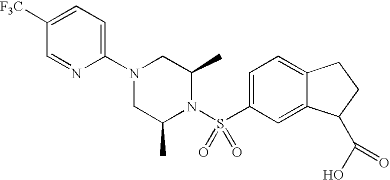 | {5-[4-(4-Trifluoromethyl-phenyl)- piperazine-1-sulfonyl]-indol-1-yl}- acetic acid |
| 32 | 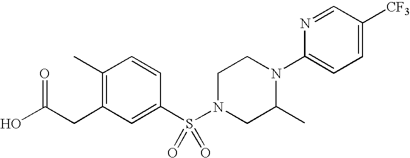 | {2-Methyl-5-[3-methyl-4-(5- trifluoromethyl-pyridin-2-yl)- piperazine-1-sulfonyl]-phenyl}-acetic acid |
| 33 | 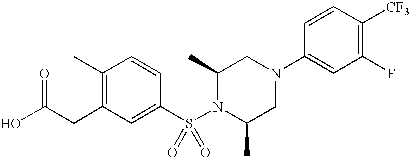 | {5-[4-(3-Fluoro-4-trifluoromethyl- phenyl)-2,6-dimethyl-piperazine-1- sulfonyl]-2-methyl-phenyl}-acetic acid |
| 34 | 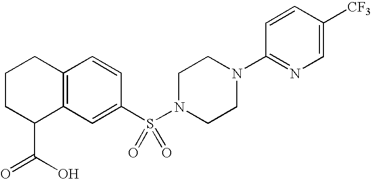 | 7-[4-(5-Trifluoromethyl-pyridin-2- yl)-piperazine-1-sulfonyl]-1,2,3,4- tetrahydro-naphthalene-1-carboxylic acid |
| 35 | 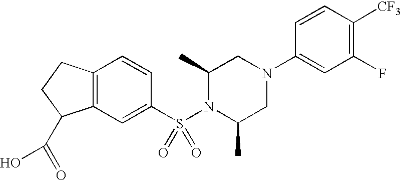 | 6-[cis-2,6-Dimethyl-4-(3-fluoro-4- trifluoromethyl-phenyl)-piperazine-1- sulfonyl]-indan-1-carboxylic acid |
| 36 | 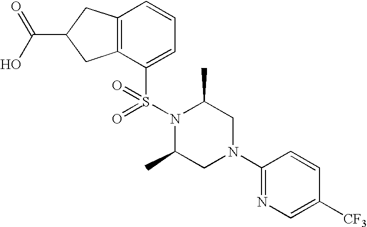 | 4-[cis-2,6-Dimethyl-4-(5- trifluoromethyl-pyridin-2-yl)- piperazine-1-sulfonyl]-indan-2- carboxylic acid |
| 37 | 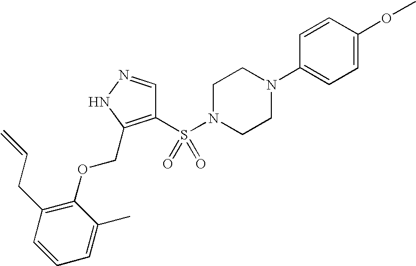 | [5-(2-Allyl-6-methyl- phenoxymethyl)-1H-pyrazol-4-yl]-[4- (4-methoxy-phenyl)-piperazin-1-yl]- methanone |
| 38 | 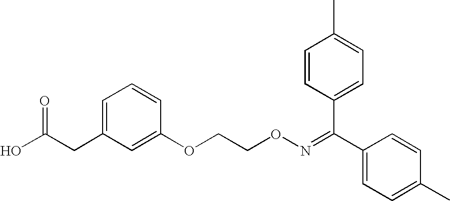 | [3-(2-Di-p-tolylmethyleneaminooxy- ethoxy)-phenyl]-acetic acid |
| 39 | 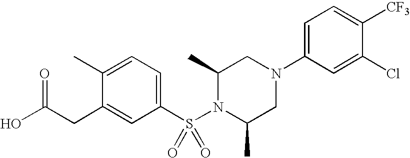 | {5-[4-(3-Chloro-4-trifluoromethyl- phenyl)-2,6-dimethyl-piperazine-1- sulfonyl]-2-methyl-phenyl}-acetic acid |
| 40 | 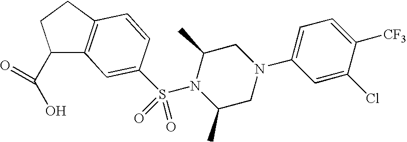 | 6-[4-(3-Chloro-4-trifluoromethyl- phenyl)-cis-2,6-dimethyl-piperazine- 1-sulfonyl]-indan-1-carboxylic acid |
| 41 | 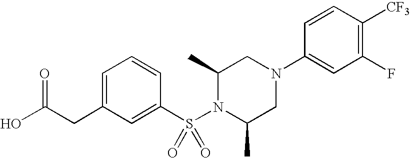 | {3-[4-(3-Fluoro-4-trifluoromethyl- phenyl)-2,6-dimethyl-piperazine-1- sulfonyl]-phenyl}-acetic acid |
| 42 | 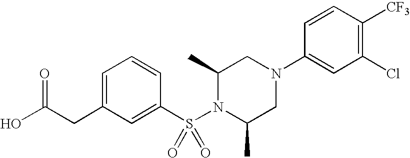 | {3-[4-(3-Chloro-4-trifluoromethyl- phenyl)-2,6-dimethyl-piperazine-1- sulfonyl]-phenyl}-acetic acid |
| 43 | 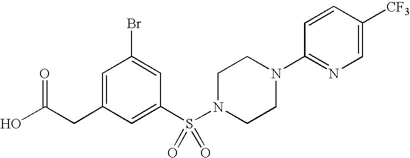 | {3-Bromo-5-[4-(5-trifluoromethyl- pyridin-2-yl)-piperazine-1-sulfonyl]- phenyl}acetic acid |
| 44 | 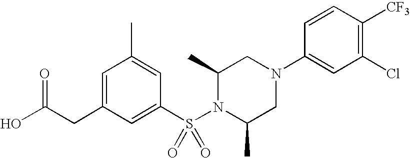 | {5-[4-(3-Chloro-4-trifluoromethyl- phenyl)-2,6-dimethyl-piperazine-1- sulfonyl]-2-methyl-phenyl}-acetic acid |
| 45 | 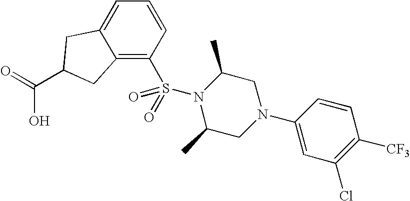 | 4-[4-(3-Chloro-4-trifluoromethyl- phenyl)-cis-2,6-dimethyl-piperazine- 1-sulfonyl]-indan-2-carboxylic acid |
| 46 | 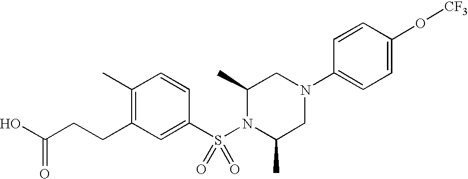 | 3-{5-[2,6-Dimethyl-4-(4- trifluoromethoxy-phenyl)-piperazine- 1-sulfonyl]-2-methyl-phenyl}- propionic acid |
| 47 | 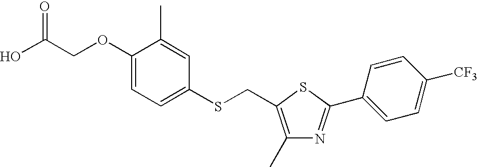 | {2-Methyl-4-[4-methyl-2-(4- trifluoromethyl-phenyl)-thiazol-5- ylmethylsulfanyl]-phenoxy}-acetic acid (GW501516; GSK, Pfizer) |
| 48 | 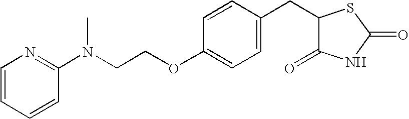 | 5-{4-[2-(Methyl-pyridin-2-yl-amino)- ethoxy]-benzyl}-thiazolidine-2,4- dione (rosiglitazone) |
| 49 | 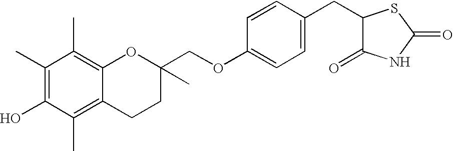 | 5-[4-(6-Hydroxy-2,5,7,8-tetramethyl- chroman-2-ylmethoxy)-benzyl]- thiazolidine-2,4-dione (troglitazone) |
| 50 | 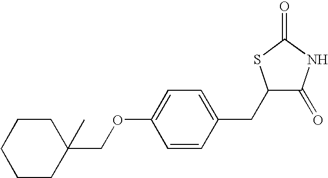 | 5-[4-(1-Methyl-cyclohexylmethoxy)- benzyl]-thiazolidine-2,4-dione (ciglitazone) |
| 51 | 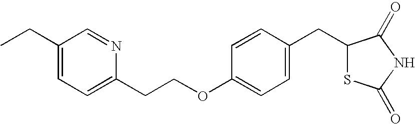 | 5-{4-[2-(5-Ethyl-pyridin-2-yl)- ethoxy]-benzyl}-thiazolidine-2,4- dione (pioglitazone; Actos, Takeda/Lilly) |
| 52 | 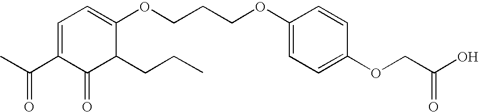 | {4-[3-(4-Acetyl-5-oxo-6-propyl- cyclohexa-1,3-dienyloxy)-propoxy]- phenoxy}-acetic acid (Merck) |

The following compounds can generally be made using the methods described above. It is expected that these compounds when made will have activity similar to those that have been made in the examples above.

### Biological Activity

All compounds listed in the examples above are known PPAR modulators. The activity of Examples 1 to 46 as PPAR modulators is demonstrated in PCT/US2004/010737, filed on Apr. 7, 2004; PCT/US2004/010889, filed on Apr. 7, 2004; PCT/US2004/010970, filed on Apr. 7, 2004; U.S. application Ser. No. 10/820,647, filed Apr. 7, 2004; PCT/US2004/043031, filed Dec. 20, 2004; PCT/US2005/011751 filed Apr. 7, 2005; U.S. application Ser. No. 11/102,356 filed Apr. 7, 2005; and U.S. application Ser. No. 11/258,463, filed Oct. 25, 2005; the contents of all of which are hereby incorporated by reference. The activity of Examples 1 through 46 were shown to be PPAR modulators by the following assay.

PPAR GAL4 Transfection Assay

Compounds may be screened for functional potency in transient transfection assays in CV-1 cells or other cell types for their ability to activate the PPAR subtypes (transactivation assay). A previously established chimeric receptor system was utilized to allow comparison of the relative transcriptional activity of the receptor subtypes on the same synthetic response element and to prevent endogenous receptor activation from complicating the interpretation of results. See, for example, Lehmann, J. M.; Moore, L. B.; Smith-Oliver, T. A; Wilkinson, W. O.; Willson, T. M.; Kliewer, S. A., An antidiabetic thiazolidinedione is a high affinity ligand for peroxisome proliferator-activated receptor □ (PPAR□), J. Biol. Chem., 1995, 270, 12953-6. The ligand binding domains for murine and human PPARα, PPARγ, and PPARδ are each fused to the yeast transcription factor GAL4 DNA binding domain. CV-1 cells were transiently transfected with expression vectors for the respective PPAR chimera along with a reporter construct containing four or five copies of the GAL4 DNA binding site driving expression of luciferase. After 8-16 h, the cells are replated into multi-well assay plates and the media is exchanged to phenol-red free DME medium supplemented with 5% delipidated calf serum. 4 hours after replating, cells were treated with either compounds or 1% DMSO for 20-24 hours. Luciferase activity was then assayed with Britelite (Perkin Elmer) following the manufacturer's protocol and measured with either the Perkin Elmer Viewlux or Molecular Devices Acquest (see, for example, Kliewer, S. A., et. al. Cell 1995, 83, 813-819). Rosiglitazone is used as a positive control in the hPPAR-γ assay. Wy-14643 and GW7647 is used as a positive control in the hPPAR-α assay. GW501516 is used as the positive control in the hPPAR-δ assay.

The activity of Examples 47 through 52 as PPAR modulators has been disclosed in U.S. Pat. No. 6,710,063, issued Mar. 23, 2004; U.S. Pat. No. 5,002,953, issued Mar. 26, 1991; U.S. Pat. No. 4,572,912, issued Feb. 25, 1986; U.S. Pat. No. 4,287,200, issued Sep. 1, 1981; U.S. Pat. No. 4,687,777, issued Aug. 18, 1987; United Kingdom Patent No. 2058785B, published Apr. 15, 1981; and PCT Application No. PCT/US97/01808, filed Jan. 31, 1997; the contents of all of which are hereby incorporated by reference.

The activity of Examples 1 to 52 as selective modulators of GPR40 is illustrated in the following assay.

GPR40 Activation Assay

Cell preparation. CHOK1/GPR40 stable cells from a single 15 cm dish (˜75% confluent) were trypsinized and counted. Cells were washed once with F12K medium containing 1% serum and then brought up to 4e5 cells/ml in the same medium (10 ml total volume). Twenty-five microliters of cells (10,000 cells) per well were then plated into a 384-well black/clear bottom plate and incubated overnight at 37° C. 10% C02.

Calcium assay. One vial of Calcium 3 Assay Reagent was reconstituted with 9 ml of Reagent buffer B and 1 ml of 25 mM probenecid in 1×HBSS/Hepes buffer (2.5 mM final concentration). Twenty-five microliters was dispensed into each well and cells were allowed to load the dye for 1 hour at room temperature. During this incubation period, the compound plate was prepared by first creating ¼ log dilutions of each compound to be tested in DMSO and then transferring 2.5 microliters of each compound into 47.5 microliters 1×HBSS/Hepes to create 5× stock compounds. Diluted compounds were transferred to a 384-well polypropylene plate and placed in the Flexstation 11384 along with one box of 384 tips.

Data acquisition and analysis. The assay plate containing the cells was placed in the Flexstation II₃₈₄ and the SoftMax Pro software was launched. The Flex mode was chosen for analysis using the following settings:

Excitation/emission 485/525 auto cutoff

PMT setting: HIGH, 2 readings

120 sec read time

Pipette height: 55 μL

Volume of compound transfer: 12.5 μL

Rate: 3 (20 μL/sec)

Data reduction: area under curve

Following the run, the data was exported to Excel and transferred to Prism Graph to plot dose-response curves and calculate the EC₅₀ values which are shown in Table 1 below.

TABLE 1
Biological Activity

| | EC₅₀, μM |
|---|---|
| | + indicates <10 |
| Example No. | − indicates >10 |
| 1 | − |
| 2 | − |
| 3 | − |
| 4 | − |
| 5 | − |
| 6 | − |
| 7 | − |
| 8 | − |
| 9 | − |
| 10 | − |
| 11 | − |
| 12 | − |
| 13 | − |
| 14 | − |
| 15 | − |
| 16 | − |
| 17 | − |
| 18 | − |
| 19 | − |
| 20 | − |
| 21 | + |
| 22 | − |
| 23 | − |
| 24 | − |
| 25 | + |
| 26 | + |
| 27 | − |
| 28 | − |
| 29 | − |
| 30 | − |
| 31 | − |
| 32 | − |
| 33 | − |
| 34 | − |
| 35 | − |
| 36 | − |
| 37 | + |
| 38 | − |
| 39 | − |
| 40 | − |
| 41 | − |
| 42 | − |
| 43 | − |
| 44 | − |
| 45 | − |
| 46 | − |
| 47 | + |
| 48 | + |
| 49 | + |
| 50 | + |
| 51 | + |
| 52 | + |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treating a PPAR-mediated disease in a patient in need thereof comprising selectively modulating PPAR over GPR40.

2. The method as recited in claim 1 wherein said treatment is of a human.

3. The method as recited in claim 1, wherein said selectivity for PPAR over GPR40 is greater than or equal to 100-fold.

4. The method as recited in claim 3, wherein said selectivity for PPAR over GPR40 is greater than or equal to 1000-fold.

5. The method as recited in claim 1 wherein said disease is a metabolic disease.

6. The method as recited in claim 5 wherein said disease is selected from the group consisting of obesity, diabetes, insulin resistance, hyperinsulinemia, hypertriglyceridemia, and glucose intolerance.

7. The method as recited in claim 1 wherein said selective modulation is additionally selective for PPARδ over other isoforms of PPAR.

8. The method as recited in claim 1, wherein said method comprises the administration of a compound which selectively modulates PPAR over GPR40.

9. The method as recited in claim 8, wherein said patient is a human.

10. The method as recited in claim 9, wherein said compound has structural Formula I: 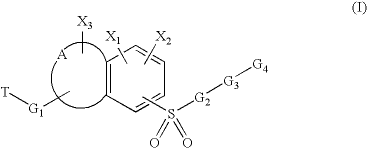
or a salt, ester, or prodrug thereof, wherein:
A is a saturated or unsaturated hydrocarbon chain or a heteroatom-comprising hydrocarbon chain having from 3 to 5 atoms, forming a five- to seven-membered ring;
T is selected from the group consisting of —C(O)OH, —C(O)NH₂, and tetrazole;
G₁ is selected from the group consisting of —(CR¹R²)ₙ—, -Z(CR¹R²)ₙ—, —(CR¹R²)ₙZ-, —(CR¹R²)ᵣZ(CR¹R²)ₛ—;
Z is O, S or NR;
n is 0, 1, or 2;
r and s are independently 0 or 1;
R¹ and R² are independently selected from the group consisting of hydrogen, halo, optionally substituted lower alkyl, optionally substituted lower heteroalkyl, optionally substituted lower alkoxy, and lower perhaloalkyl or together may form an optionally substituted cycloalkyl;
X₁, X₂, and X₃ are independently selected from the group consisting of hydrogen, optionally substituted lower alkyl, optionally substituted cycloalkyl, halogen, perhaloalkyl, hydroxy, optionally substituted lower alkoxy, nitro, cyano, and NH₂;
G₂ is selected from the group consisting of a saturated or unsaturated cycloalkyl or heterocycloalkyl linker, optionally substituted with X₄ and X₅;
X₄ and X₅ are independently selected from the group consisting of hydrogen, optionally substituted lower alkyl, halogen, lower perhaloalkyl, hydroxy, optionally substituted lower alkoxy, nitro, cyano, NH₂, and CO₂R, or X₄ and X₅ together may form a carbocycle;
R is selected from the group consisting of optionally substituted lower alkyl and hydrogen;
G₃ is selected from the group consisting of a bond, a double bond, —(CR³R⁴)ₘ—, carbonyl, and —(CR³R⁴)ₘCR³═CR⁴—;
m is 0, 1, or 2;
R³ and R⁴ are independently selected from the group consisting of hydrogen, optionally substituted lower alkyl, optionally substituted lower alkoxy, optionally substituted aryl, lower perhaloalkyl, cyano, and nitro;
G₄ is selected from the group consisting of hydrogen, optionally substituted aryl, optionally substituted heteroaryl, optionally substituted cycloalkyl, optionally substituted cycloheteroalkyl, optionally substituted cycloheteroaryl, optionally substituted cycloalkenyl, and N═(CR⁵R⁶); and
R⁵ and R⁶ are independently selected from the group consisting of hydrogen, optionally substituted alkyl, optionally substituted aryl, optionally substituted heteroaryl, optionally substituted cycloalkyl, optionally substituted cycloalkenyl, and optionally substituted cycloheteroalkyl.

11. The method as recited in claim 10 wherein said compound is selected from the group consisting of Examples 1 to 46, or a salt thereof.

12. The method as recited in claim 11 wherein said compound is selected from the group consisting of 4-[cis-2,6-dimethyl-4-(4-trifluoromethoxy-phenyl)-piperazine-1-sulfonyl]-indan-2-carboxylic acid and 4-[cis-2,6-dimethyl-4-(4-trifluoromethoxy-benzyl)-piperazine-1-sulfonyl]-indan-2-carboxylic acid.

13. The method as recited in claim 12 wherein said compound is selected from the group consisting of (S)-4-[cis-2,6-dimethyl-4-(4-trifluoromethoxy-phenyl)-piperazine-1-sulfonyl]-indan-2-carboxylic acid tosylate and (S)-4-[cis-2,6-dimethyl-4-(4-trifluoromethoxy-benzyl)-piperazine-1-sulfonyl]-indan-2-carboxylic acid tosylate.

14. The method as recited in claim 9, wherein said compound is administered to a human in a dose greater than or equal to 10 mg.

15. The method as recited in claim 14, wherein said compound is administered to a human in a dose greater than or equal to 20 mg.

16. The method as recited in claim 15, wherein said compound is administered to a human in a dose greater than or equal to 40 mg.

17. The method as recited in claim 16, wherein said compound is administered to a human in a dose greater than or equal to 60 mg.

18. The method as recited in claim 17, wherein said compound is administered to a human in a dose greater than or equal to 80 mg.

19. The method as recited in claim 18, wherein said compound is administered to a human in a dose greater than or equal to 100 mg.

20. The method as recited in claim 9, wherein said compound is administered to a human in a dose which is therapeutically effective.

21. The method as recited in claim 9 wherein said disease is a metabolic disease.

22. The method as recited in claim 21 wherein said disease is selected from the group consisting of obesity, diabetes, insulin resistance, hyperinsulinemia, hypertriglyceridemia, and glucose intolerance.

23. The method as recited in claim 8, wherein said selectivity for PPAR over GPR40 is greater than or equal to 100-fold.

24. The method as recited in claim 23, wherein said selectivity for PPAR over GPR40 is greater than or equal to 1000-fold.

25. The method as recited in claim 9, wherein said compound is an analog of a known PPAR modulator, structurally modified to have selectivity over GPR40.

26. The method as recited in claim 25, wherein said known PPAR modulator is selected from the group consisting of glitazones, glitazars, and fibrates.

27. The method as recited in claim 26, wherein said glitazone is selected from the group consisting of rosiglitazone, ciglitazone, pioglitazone, troglitazone, netoglitazone, and isaglitazone.

28. The method as recited in claim 26, wherein said glitazar is selected from the group consisting of muraglitazar, naveglitazar, and tesaglitazar.

29. The method as recited in claim 26, wherein said fibrate is selected from the group consisting of benzafibrate, clofibrate, ciprofibrate, etofibrate, fenofibrate, gemflbrozil, gefitinib, and GW5907354.

30. The method as recited in claim 25, wherein said known PPAR modulator is selected from the group consisting of GW501516, MC-555, GSK677954, and GSK625019.

31. A method of reducing or eliminating one or more side effects associated with a modulator of PPAR, comprising selectively modulating PPAR over GPR40.

32. The method as recited in claim 31 wherein said side effect is selected from the group consisting of hyperinsulinemia, hepatic steatosis, hypertriglyceridemia, and glucose intolerance.

33. A method of increasing HDLs (high-density lipoproteins) or HDL-C (high density lipoprotein cholesterol) without causing a hypoglycemic state comprising selectively modulating PPAR over GPR40.

34. The method as recited in claim 33 wherein said increase in HDL or HDL-C is not accompanied by induction of a hypoglycemic state.

35. A method of reducing triglycerides without causing a hypoglycemic state comprising selectively modulating PPAR over GPR40.

36. The method as recited in claim 35 wherein said reduction in triglycerides is not accompanied by induction of a hypoglycemic state.

37. A method of reducing visceral fat in a patient in need thereof, comprising selectively modulating PPAR over GPR40.

38. The method as recited in claim 37 wherein said visceral fat is reduced selectively over other types of fat.

39. A method of reducing insulin resistance in a patient in need thereof, comprising selectively modulating PPAR over GPR40.

40. The method as recited in claim 39 wherein said modulation is additionally selective for PPARδ.

41. A method of enhancing glucose utilization in a patient in need thereof, comprising selectively modulating PPAR over GPR40.

42. The method as recited in claim 41 wherein said modulation is additionally selective for PPARδ.
